(19) United States
(12) Patent Application Publication
(10) Pub. No.: US 2005/0112604 A1
(43) Pub. Date: May 26, 2005

(54) Loss of heterozygosity of the DNA markers in the 12q22-23 region

(76) Inventors: Akihide Fujimoto, Kanazawa (JP); Dave S.B. Hoon, Los Angeles, CA (US)

Correspondence Address: HOGAN & HARTSON L.L.P., 500 S. GRAND AVENUE, SUITE 1900, LOS ANGELES, CA, 90071-2611, US

(21) Appl. No.: 10/801,956

(22) Filed: Mar. 15, 2004

Related U.S. Application Data
(60) Provisional application No. 60/455,006, filed on Mar. 14, 2003.

(51) Int. Cl. C12Q 1/68

(52) U.S. Cl. 435/6

(57) ABSTRACT

A method of detecting DNA markers in the 12q22-23 region. The method comprises providing a sample containing acellular DNA from a subject and detecting one or more DNA markers in the 12q22-23 region in the sample. Also disclosed are methods of diagnosing and monitoring cancer; methods of determining the efficacy of a therapy, and the probabilities of survival and responsiveness to a therapy; and packaged products for using these methods.

### RELATED APPLICATION

This application claims priority to U.S. Provisional Application Ser. No. 60/455,006, filed Mar. 14, 2003, the content of which is incorporated herein by reference.

### FIELD OF THE INVENTION

The present invention relates generally to the fields of molecular biology and oncology. In particular, this invention relates to detection of loss of heterozygosity (LOH) of DNA markers in the 12q22-23 region, and use of these DNA markers for detecting and treating cancer.

### BACKGROUND OF THE INVENTION

APAF-1 is an essential downstream target of p53 in the intrinsic apoptotic pathway (Soengas et al., 1999, Science 284:156-159; Soengas et al., 2001, Nature 409:207-211; Moroni et al., 2001, Nat. Cell Biol. 3:552-558; and Robles et al., 2001, Cancer Res. 61:6660-6664). Activated p53 is a transcriptional transactivator of genes and targets APAF-1 by the following pathway: p53 controls the release of cytochrome c from mitochondria during apoptosis (Robles et al., 2001, Cancer Res. 61:6660-6664; Mihara et al., 2003, Mol. Cell. 11:577-590; Fortin et al., 2001, J. Cell Biol. 155:207-216; and Moroni et al., 2001, Nat. Cell Biol. 3:552-558). In the presence of cytochrome c, APAF-1 can bind to procaspase 9, forming an apoptosome. Activation of caspase 9 in the apoptosome results in activation of downstream caspases such as 3, 6, and 7 (Li et al., 1997, Cell 91:479-489).

APAF-1 was originally shown to be located at chromosome loci 12q22-23, and frequent loss of heterozygosity in this region has been reported in male germ cell tumors (Murty et al., 1996, Genomics. 35:562-570; Murty and Chaganti, 1998, Semin. Oncol. 25:133-144; and Murty et al., 1999, Genome Res. 9:662-671) and pancreatic, ovarian, and gastric carcinomas (Kimura et al., 1996, Genes Chromosomes Cancer 17:88-93; Kimura et al., 1998, Cancer Res. 58:2456-2460; Yatsuoka et al., 2000, Am. J. Gastroenterol. 95:2080-2085; Hatta et al., 1997, Br. J. Cancer 75:1256-1262; and Schneider et al., 2003, Mol. Pathol. 56:141-149). Recently, Soengas et al. (Soengas et al., 2001, Nature 409:207-211) demonstrated LOH on the APAF-1 gene locus (12q22-23) of 10 of 24 (42%) metastatic melanomas and that LOH was associated with loss of APAF-1 mRNA expression.

Since the time of publication of the study by Soengas et al., there has been a significant reassessment of the APAF-1 gene location. New data published in the National Center for Biotechnology Information (NCBI) database indicates that the APAF-1 gene is more distant (>0.3Mb) to the centromere on chromosome 12q. This significant change must be considered in lieu of previous reports which have used a different location. Because of such, APAF-1 gene status by LOH analysis of this region mandates reanalysis.

The role of APAF-1 in other cancers has not been well studied. In leukemia, APAF-1 status has been examined as a prognostic factor; no correlation was demonstrated between APAF-1 expression level and the response to chemotherapy in acute leukemia (Svingen et al., 2000, Blood 96:3922-3931). However, no major reports or detailed studies have examined allelic imbalance in the 12q22-23 region of primary and metastatic melanoma, and no correlative studies of APAF-1 status with the progression and prognosis of cutaneous melanoma exist.

Recently, the concurrent administration of biochemotherapy (BC) has shown improvement in response in AJCC stage IV melanoma patients (O'Day et al., 1999, J. Clin. Oncol. 17:2752-2761; O'Day et al., 2002, Clin. Cancer Res. 8:2775-2781; Atkins et al., 2002, Clin. Cancer Res. 8:3075-3081; McDermott et al., 2000, Clin. Cancer Res. 6:2201-2208; and Legha et al., 1998, J. Clin. Oncol. 16:1752-1759). However, as with any treatment regimen, it is difficult to predict patient response. Identification of molecular predictors of therapeutic response may permit a more efficient utilization and improve stratification of design strategies.

### SUMMARY OF THE INVENTION

This invention is based on the unexpected discovery that LOH of DNA markers in the 12q22-23 region can be detected in acellular samples, and that the LOH of these DNA markers can be used for cancer diagnosis, monitoring and prognosis.

Accordingly, the invention features a method of detecting DNA markers in the 12q22-23 region. The method involves providing a sample containing acellular DNA from a subject and detecting one or more DNA markers in the 12q22-23 region in the sample. The acellular sample may be, e.g., a serum sample or a plasma sample. Examples of the DNA markers include D12S1657, D12S393, D12S1706, D12S346, and a combination thereof (i.e., a combination of any two or three of the markers, or a combination of all of the four markers). In a preferred embodiment, the DNA markers are associated with the APAF-1 gene, i.e., the presence or absence of the marker indicates the presence or absence of the APAF-1 gene.

DNA markers in the 12q22-23 region are useful for cancer diagnosis, monitoring and prognosis. In one aspect, the invention features a method of detecting cancer, e.g., melanoma, colon cancer, breast, and brain cancer. The method involves providing a sample containing acellular DNA from a subject and detecting one or more DNA markers in the 12q22-23 region in the sample, wherein LOH of the DNA markers is indicative of cancer, e.g., a cancer at the primary or metastatic stage.

In another aspect, the invention features a method of staging cancer. The method involves providing a sample containing acellular DNA from a subject suffering from cancer and detecting one or more DNA markers in the 12q22-23 region in the sample, wherein LOH of the DNA markers indicates a high probability of a metastatic cancer.

In still another aspect, the invention features a method of monitoring progression of cancer. The method involves providing a sample containing acellular DNA from a subject suffering from cancer and detecting one or more DNA markers in the 12q22-23 region in the sample, wherein LOH of the DNA markers indicates a high probability of a progressing cancer.

In yet another aspect, the invention features a method of determining the efficacy of a cancer therapy (e.g., a chemotherapy, radiation therapy, gene therapy, immunotherapy, surgical procedure, or a combination thereof). The method involves providing a sample containing acellular DNA from a subject suffering from cancer and administered with a therapy and detecting one or more DNA markers in the 12q22-23 region in the sample, wherein LOH of the markers indicates poor efficacy of the therapy.

The invention is also based on the unexpected discovery that DNA markers in the 12q22-23 region are useful prognostic predictors for disease outcomes and responses to therapies. Therefore, the invention provides a method of determining the probability of survival, comprising providing a sample from a subject suffering from a metastatic cancer and detecting one or more DNA markers in the 12q22-23 region in the sample, wherein LOH of the markers indicates a low probability of survival. The sample may be, e.g., a tumor sample, a serum sample, or a plasma sample. The cancer may be melanoma, e.g., a stage III melanoma such as an RLM (regional lymph node metastasis) melanoma or an ITM (in-transit metastasis) melanoma, or a stage IV melanoma. Other examples of cancers include colon cancer, breast cancer, and brain cancer.

The invention further provides a method of determining the probability of responsiveness to a therapy, comprising providing a sample from a subject suffering from cancer and detecting one or more DNA markers in the 12q22-23 region in the sample, wherein LOH of the markers indicates a low probability of responsiveness to a therapy. The cancer may be melanoma, colon cancer, breast cancer, brain cancer, or other cancer. The melanoma may be, e.g., a metastatic melanoma such as a stage III melanoma or a stage IV melanoma.

The invention also provides a packaged product, comprising a container, one or more agents for detecting one or more DNA markers at the 12q22-23 region in a sample, and an insert associated with the container. In one embodiment, the insert indicates that the sample contains acellular DNA. In another embodiment, the sample is from a subject suffering from a metastatic cancer, and the insert indicates that LOH of the markers indicates a low probability of survival. In still another embodiment, the sample is from a subject suffering from cancer, and the insert indicates that LOH of the markers indicates a low probability of responsiveness to a therapy.

In summary, the invention provides cancer diagnosing and monitoring methods. DNA markers in the 12q22-23 region can be used as genomic surrogates of disease outcome for cancer patients. Detection of these DNA markers in acellular samples enables diagnosing, monitoring and prognosing cancer without direct tumor sampling.

The above-mentioned and other features of this invention and the manner of obtaining and using them will become more apparent, and will be best understood, by reference to the following description, taken in conjunction with the accompanying drawings. These drawings depict only typical embodiments of the invention and do not therefore limit its scope.

### BRIEF DESCRIPTION OF THE FIGURES

FIG. 1 is a representative electrophoregram analysis of primary and metastatic melanomas demonstrating LOH at microsatellite markers D12S1657 and D12S393.

FIG. 2 shows LOH on APAF-1 locus (chromosome 12q22-23) between matched primary and metastatic melanoma tumors.

FIG. 3 shows correlation between APAF-1 LOH and mRNA expression level in 22 melanoma tumors.

FIG. 4 shows correlation between survival and (A) APAF-1 LOH in primary melanoma, (B) APAF-1 LOH in AJCC stage III/IV metastatic melanoma, and (C) allelic imbalance between D12S1657 and D12S393 of AJCC stage III/IV metastatic melanoma.

FIG. 5 shows correlation between survival and APAF-1 LOH in AJCC stage III melanoma (A), AJCC stage III melanoma with RLM (B) AJCC stage III melanoma with ITM (C).

FIG. 6 shows allelic imbalance (Al) on 12q22-23 in pre-BC and post-BC sera.

FIG. 7 shows results of AI on 12q22-23 for all sera.

FIG. 8a shows correlation of AI on 12q22-23 in serum with overall survival.

FIG. 8b shows correlation of BC response with overall survival.

### DETAILED DESCRIPTION OF THE INVENTION

Cancer cells almost invariably undergo loss of genetic material (DNA) when compared to normal cells. This deletion of genetic material which almost all, if not all, varieties of cancer undergo is referred to as “loss of heterozygosity” (LOH). The loss of genetic material from cancer cells can result in the selective loss of one of two or more alleles of a gene vital for cell viability or cell growth at a particular locus on the chromosome. All genes, except those of the two sex chromosomes, exist in duplicate in human cells, with one copy of each gene (allele) found at the same place (locus) on each of the paired chromosomes. Each chromosome pair thus contains two alleles for any gene, one from each parent. This redundancy of allelic gene pairs on duplicate chromosomes provides a safety system. If a single allele of any pair is defective or absent, the surviving allele will continue to produce the coded protein.

Due to the genetic heterogeneity or DNA polymorphism, many of the paired alleles of genes differ from one another. When the two alleles are identical, the individual is said to be homozygous for that pair of alleles at that particular locus. Alternatively, when the two alleles are different, the individual is heterozygous at that locus. Typically, both alleles are transcribed and ultimately translated into either identical proteins in the homozygous case or different proteins in the heterozygous case. If one of a pair of heterozygous alleles is lost due to deletion of DNA from one of the paired chromosomes, only the remaining allele will be expressed and the affected cells will be functionally homozygous. This situation is termed as “loss of heterozygosity” (LOH) or reduction to homozygosity. Following this loss of an allele from a heterozygous cell, the protein or gene product thereafter expressed will be homogeneous because all of the protein will be encoded by the single remaining allele. The cell becomes effectively homozygous at the gene locus where the deletion occurred. Almost all, if not all, cancer cells undergo LOH at some chromosomal regions.

Through the use of DNA probes, DNA from an individual's normal cells can be compared with DNA extracted from the same individual's tumor cells and LOH can be identified using experimental techniques well known in the art. Alternatively, LOH can be assayed by demonstrating two polymorphic forms of a protein in normal heterozygous cells, and only one form in cancer cells where the deletion of an allele has occurred. See, for example, Lasko et al, 1991, Annu. Rev. Genet. 25:281-314.

Recent advances in molecular biology have revealed that genesis and progression of tumors follow an accumulation of multiple genetic alterations, including inactivation of tumor suppressor genes and/or activation of proto-oncogenes. There are over 40 known proto-oncogenes and suppressor genes to date, which fall into various categories depending on their functional characteristics. These include, growth factors and growth factor receptors, messengers of intracellular signal transduction pathways, for example, between the cytoplasm and the nucleus, and regulatory proteins influencing gene expression and DNA replication. Frequent LOH on specific chromosomal regions has been reported in many kinds of malignancies, which indicates the existence of putative tumor suppresser genes or tumor-related genes on or near these loci. LOH analysis is a powerful tool to search for a tumor suppresser gene by narrowing and identifying the region where a putative gene exists. By now, numerous LOH analyses, combined with genetic linkage analysis on pedigrees of familial cancer (Vogelstein et al, 1988, New England Journal of Medicine 319(9):525-532; Fearon et al., 1990, Cell 61:759-767; and Friend et al., 1986, Nature 323:643-646) or homozygous deletion analyses (Call et al., 1990, Cell 60:509-520; Kinzler et al., 1991, Science 253:661-665; and Baker et al., 1989, Science 244:217-221) have identified many kinds of candidate tumor suppressor or tumor-related genes. Also, because allelic losses on specific chromosomal regions are the most common genetic alterations observed in a variety of malignancies, microsatellite analysis has been applied to detect DNA of cancer cells in specimens from body fluids, such as sputum for lung cancer and urine for bladder cancer (Rouleau et al., 1993, Nature 363:515-521; and Latif et al., 1993, Science 260:1317-1320). Moreover, it has been established that markedly increased concentrations of soluble DNA are present in plasma of individuals with cancer and some other diseases, indicating that cell free serum or plasma can be used for detecting cancer DNA with microsatellite abnormalities (Kamp et al., 1994, Science 264:436-440; and Steck et al., 1997, Nature Genetics 15:356-362). Two groups have reported microsatellite alterations in plasma or serum of a limited number of patients with small cell lung cancer or head and neck cancer (Hahn et al., 1996, Science 271:350-353; and Miozzo et al., 1996, Cancer Research 56:2285-2288).

Recent developments in cancer therapeutics have demonstrated the need for more sensitive staging and monitoring procedures to ensure initiation of appropriate treatment, to define the end points of therapy and to develop and evaluate novel treatment modalities and strategies. In the management of melanoma patients, the choice of appropriate initial treatment depends on accurate assessment of the stage of the disease. Patients with limited or regional disease generally have a better prognosis and are treated differently than patients who have distant metastases (Minna et al., 1989, Cancer Principals and Practices of Oncology, DeVita et al., ed., Lippincott, Philadelphia 591-705). However, conventional techniques to detect these metastases are not very sensitive, and these patients are often not cured by primary tumor resection because they have metastases that are not identified by standard methods during preoperative staging. Thus, more sensitive methods to detect metastases in other types of carcinomas would identify patients who will not be cured by local therapeutic measures, for whom effective systemic therapies would be more appropriate.

The strategy of the present invention is to utilize genetic differences between normal and cancer cells for diagnosis and monitoring of melanoma patients. Many genes coding for proteins or other factors vital to cell survival and growth that are lost, can be identified through LOH analysis of microsatellite and single nucleotide polymorphism (SNP) loci in cancer cells and mapped to specific chromosomal regions. In melanoma, mutations of several already-known tumor suppresser genes such as p53 gene, neurofibromatosis 1 (NF1) gene, and NF2 gene have been reported at a low frequency and deletions and/or mutations of the cyclin dependent kinase 4 (CDK4) inhibitor gene, which is a responsible tumor suppresser gene for a familial melanoma, have been thought to be important genetic changes in tumor development (Miozzo et al., 1996, Cancer Research 56:2285-2288). In addition to the locus of CDK4 inhibitor gene (9p21), frequent chromosomal deletions have been reported on 1p36, 3p25, 6q22-q26, 10q24-q26, and 11q23. (Mao et al, 1996, Science 271:659-662; Stroun et al., 1987, Eur. J. Cancer Clin. Oncol. 23(6):707-712; Chen et al., 1996, Nature Medicine 2(9):1033-1035; and Nawroz et al., 1996, Nature Medicine 2(9):1035-1037). An efficient method of testing DNA microsatellite loci for LOH allows early diagnosis of melanoma patients and monitoring of the progression of the disease as well as effectiveness of the therapeutic regimen.

Cutaneous melanoma is a highly aggressive tumor that is relatively resistant to chemotherapy and radiotherapy. This resistance may be in part due to inhibition of apoptosis. Apoptotic protease activating factor-1 (APAF-1), a candidate tumor suppressor gene, mediates p53-induced apoptosis, and its loss promotes oncogenic transformation. To determine if loss of the APAF-1 locus influences tumor progression, we assessed LOH of microsatellites on the APAF-1 locus (12q22-23) in 62 primary and 112 metastatic melanomas. We discovered that frequency of allelic imbalance was significantly higher in metastatic tumors (n=36/98, 37%) than in primary melanomas (n=10/54, 19%) (P=0.02). In metastatic melanomas, APAF-1 loss significantly correlated with a worse prognosis (P<0.05) in the patients and its loss during melanoma tumor progression suggests that APAF-1 is a tumor suppressor gene. Furthermore, LOH was frequent in the 12q22-23 chromosome region centromeric to the APAF-1 locus, suggesting that other tumor-related genes may be present in the 12q22-23 region. In summary, the study demonstrates that allelic imbalance in the 12q22-23 region is a genomic surrogate of poor disease outcome for cutaneous melanoma patients.

We also evaluated allelic imbalance (AI) on 12q22-23 in serum DNA to predict BC treatment response. Sera were collected from 49 AJCC stage IV melanoma patients treated with BC. Frequency of AI of the 12q22-23 region was 36%. Responders showed a significantly lower frequency of AI (5 of 24, 21%) compared to non-responders (11 of 20, 55%) (Fisher's exact test P<0.029). AI on 12q22-23 in serum was associated with worse prognosis (log-rank test P<0.046). These findings indicate that tumor related AI on 12q22-23 in serum may have clinical utility in predicting tumor resistance to therapy without direct tumor sampling.

It is an object of the invention to provide a method of detecting DNA markers in the 12q22-23 region. This method comprises the steps of (1) providing from a subject a sample containing acellular DNA, and (2) detecting one or more DNA markers in the 12q22-23 region in the sample.

Acellular DNA can be obtained from a sample of a biological fluid by deproteinizing the sample and extracting DNA according to the procedures well known in the art. Examples of biological fluids include urine, blood plasma or serum, sputum, cerebral spinal fluid, peritoneal fluid, ascites fluid, saliva, and stools. The DNA to be tested may be a fraction of a larger molecule or can be present initially as a discrete molecule. Where the test DNA contains two strands, it may be necessary to separate the strands of the nucleic acid before it can be used, e.g., as a template for amplification. Strand separation can be effected either as a separate step or simultaneously with synthesis of primer extension products. This strand separation can be accomplished using various suitable denaturing conditions, including physical, chemical, or enzymatic means. If the nucleic acid is single stranded, its complement is synthesized by adding one or two oligonucleotide primers. If a single primer is utilized, a primer extension product is synthesized in the presence of primer, an agent for polymerization, and the four nucleoside triphosphates. The product will be complementary to the single-stranded nucleic acid and will hybridize with a single-stranded nucleic acid to form a duplex of unequal length strands that may then be separated into single strands to produce two single separated complementary strands.

A DNA marker refers to a DNA sequence (e.g., a microsatellite or SNP locus) associated with a specific biological event (e.g., presence or absence of a gene, expression of a gene, and occurrence of a disease). Microsatellites are short repetitive sequences of DNA widely distributed in the human genome. Somatic alterations in the repeat length of such microsatellites have been shown to represent a characteristic feature of tumors. SNP is a common nucleotide variant in DNA at a single site. Each individual has many single nucleotide polymorphisms that together create a unique DNA sequence. In a preferred embodiment, the DNA markers include D12S1657, D12S393, D12S1706, or D12S346. In other embodiments, other DNA markers in the 12q22-23 region may be used. These markers can be tested either independently or in combination with each other, or with markers beyond the 12q22-23 region (e.g., D9S157). Preferably, these DNA markers are associated with the APAF-1 gene.

Detection of a DNA marker can be accomplished by a number of means well known in the art. One means of detecting a DNA marker is by digesting a test DNA sample with a restriction endonuclease. Restriction endonucleases are well known in the art for their ability to cleave DNA at specific sequences, and thus generate a discrete set of DNA fragments from each DNA sample. The restriction fragments of each DNA sample can be separated by any means known in the art. For example, agarose or polyacrylamide gel electrophoresis can be used to electrophoretically separate fragments according to physical properties such as size. The restriction fragments can be hybridized to nucleic acid probes which detect restriction fragment length polymorphisms (RFLP). There are various hybridization techniques known in the art, including both liquid and solid phase techniques. One particularly useful method employs transferring the separated fragments from an electrophoretic gel matrix to a solid support such as nylon or filter paper so that the fragments retain the relative orientation which they had on the electrophoretic gel matrix. The hybrid duplexes can be detected by any means known in the art, for example, by autoradiography if the nucleic acid probes have been radioactively labeled. Other labeling and detection means are well known in the art and may be used accordingly.

An alternative means for detecting a DNA marker is by using PCR (polymerase chain reaction; see, e.g., U.S. Pat. Nos. 4,683,195, 4,683,202, and 4,683,194). This method allows amplification of discrete regions of DNA containing microsatellite sequences. Amplification is accomplished by annealing, i.e., hybridizing a pair of single stranded primers, usually comprising DNA, to a target DNA. The primers embrace oligonucleotides of sufficient length and appropriate sequence so as to provide specific initiation of polymerization of a significant number of nucleic acid molecules containing the target nucleic acid. In this manner, it is possible to selectively amplify the specific target nucleic acid sequence containing the nucleic acid of interest. More specifically, the primers are designed to be substantially complementary to each strand of target nucleotide sequence to be amplified. Substantially complementary means that the primers must be sufficiently complementary to hybridize with their respective strands (i.e., with the flanking sequences) under conditions which allow amplification of the nucleotide sequence to occur. The primer is preferably single stranded for maximum efficiency in amplification but may be double-stranded. If double-stranded, the primer is first treated to separate its strands before being used to prepare extension products. Preferably, the primer is an oligodeoxyribonucleotide. The primer must be sufficiently long to prime the synthesis of extension products in the presence of the inducing agent for polymerization. The exact length of a primer will depend on many factors, including temperature, buffer, and nucleotide composition. The oligonucleotide primers for use in the present invention may be prepared using any suitable method, such as conventional phosphotriester and phosphodiester methods or automated embodiments thereof. In one such automated embodiment, diethylphosphoramidites are used as starting materials and may be synthesized as described by Beaucage et al. (Tetrahedron Letters 22:1859-1862, 1981). One method for synthesizing oligonucleotides on a modified solid support is described in U.S. Pat. No. 4,458,066. The primers are annealed to opposite strands of the DNA sequence containing a DNA marker, such that they prime DNA synthesis in opposite but convergent directions on a chromosome. Amplification of the region containing the DNA marker is accomplished by repeated cycles of DNA synthesis. Experimental conditions conducive to synthesis include the presence of nucleoside triphosphates and an agent for polymerization, such as DNA polymerase, and a suitable temperature and pH. Preferably, the DNA polymerase is Taq polymerase which is relatively heat insensitive. The amplification procedure includes a specified number of cycles of amplification in a DNA thermal cycler. After an initial denaturation period of 5 minutes, each amplification cycle preferably includes a denaturation period of about 1 minute at 95° C., primer annealing for about 2 minutes at 58° C., and an extension at 72° C. for approximately 1 minute. Following the amplification, aliquots of amplified DNA from the PCR can be analyzed by techniques such as electrophoresis through agarose gel using ethidium bromide staining. Improved sensitivity may be attained by using labeled primers and subsequently identifying the amplified product by detecting radioactivity or chemiluminescense on film.

In a preferred embodiment, the assay involves labeling of the PCR primers with multiple types of chromophore dyes. In another embodiment, the PCR primers are labeled with an atom or inorganic radical, most commonly using radionuclides, but also perhaps heavy metals. Radioactive labels include ³²P, ¹²⁵I, ³H, ¹⁴C, or any radioactive label which provides for an adequate signal and has sufficient half-life. Other labels include ligands, which can serve as a specific binding pair member for a labeled ligand, and the like.

Another object of the invention is to provide a method of detecting LOH in biological fluids, wherein the presence of LOH is associated with the occurrence of cancer. This method represents a significant advance over such techniques as tissue biopsy by providing a non-invasive, rapid, and accurate method for detecting LOH of specific alleles associated with cancer. Thus, the present invention provides a method which can be used to screen high-risk populations and to monitor high risk patients undergoing chemoprevention, chemotherapy, immunotherapy, surgical procedure, or other treatment.

For detection of cancer, a sample containing acellular DNA is obtained from a subject and one or more DNA markers in the 12q²²-²3 region is analyzed. LOH of the DNA markers indicates that the subject is suffering from cancer or at risk of developing cancer.

According to the method of the present invention, DNA is isolated from a biological fluid of a patient. For comparison, a control DNA sample may be prepared, for example, from a non-neoplastic tissue from the same patient, or from a biological fluid or tissue from a normal person. It is desirable that the alleles used in the allelotype loss analysis be those for which the subject is heterozygous. Determination of heterozygosity is well within the skill of the art. Loss of an allele is ultimately determined by comparing the pattern of bands corresponding to the allele in the control sample to the test sample and noting the size, number of bands, or level of amplification of signal of individual bands. For example, LOH may be defined when one allele showed ≧40% reduction of peak intensity for serum DNA as compared to the corresponding allele identified in the control DNA (see Example 1 below).

Another object of the invention is to provide methods for identifying and assessing the extent of genetic change in biological fluids. More specifically, the present invention provides methods for staging cancer patients by detecting the loss of a specified set of polymorphic alleles (LOH), alone or in combination, in DNA from biological fluids. The steps of the method include obtaining a sample containing acellular DNA from a subject suffering from cancer and detecting one or more DNA markers in the 12q22-23 region in the sample. LOH of the DNA markers indicates that the subject has a high probability of suffering from a metastatic cancer.

This invention also provides a logistically practical assay to monitor the genetic changes during cancer progression. The events of tumor progression are dynamic and the genetic changes that concurrently occur also are very dynamic and complex. The most significant advantage of this approach compared to other approaches is the ability to monitor disease progression and genetic changes without assessing the tumor. This is particularly important during early phases of distant disease spread, in which subclinical disease is undetectable by conventional imaging techniques. In addition, in advance stage diseases or inoperable sites in which tumor tissue is very difficult or impossible to obtain for genetic analysis, the present invention provides an alternative for assessing LOH. To monitor the progression of a cancer, an acellular DNA sample is isolated from a subject suffering from cancer, and one or more DNA markers in the 12q22-23 region are detected. LOH of the DNA markers indicates that the subject is likely to have a progressing cancer.

The invention further provides a method of determining the efficacy of a cancer therapy. A therapy is administered to a patient suffering from cancer, and a biological fluid is obtained from the patient. Acellular DNA is isolated from the fluid, and one or more DNA markers in the 12q22-23 region are detected. LOH of the markers indicates that the efficacy of the therapy is poor.

Because the methods described above require only DNA extraction from bodily fluid such as blood, it can be performed at any time and repeatedly on a single patient. Blood can be taken and monitored for LOH before or after surgery; before, during, and after treatment, such as chemotherapy, radiation therapy, gene therapy or immunotherapy; or during follow-up examination after treatment for disease progression, stability, or recurrence. The method of the present invention also may be used to detect subclinical disease presence or recurrence with an LOH marker specific for that patient since LOH markers are specific to an individual patient's tumor. The method also can detect if multiple metastases may be present using tumor specific LOH markers.

Further, the invention provides predictive measures of response to cancer therapies and mortality.

More specifically, the invention provides a method of predicting the probability of survival of a subject suffering from a metastatic cancer. The method comprises providing a sample from the subject and detecting one or more DNA markers in the 12q22-23 region. If LOH of the markers occurs, the subject is expected to have a low probability of survival. For example, in the case of melanoma, patients with a stage III melanoma (e.g., RLM or ITM) or a stage IV melanoma, the survival rate is lower for LOH positive patients than that for LOH negative patients.

In one embodiment, the sample is a sample of a biological fluid. In another embodiment, the sample is a tumor sample. For a tumor sample, if a non-neoplastic tissue is used as a control sample, it can be of the same type as the neoplastic tissue or from a different organ source. It is desirable that the neoplastic tissue contains primarily neoplastic cells and that normal cells be separated from the neoplastic tissue. Ways for separating cancerous from non-cancerous cells are known in the art and include, for example, microdissection of tumor cells from normal cells of tissues, DNA isolation from paraffin-embedded sections and cryostat sections, as well as flow cytometry to separate aneuploid cells from diploid cells. DNA can also be isolated from tissues preserved in paraffin. Separations based on cell size or density may also be used. Once the tissues have been microdissected, DNA can be isolated from the tissue using any means known in the art. Frozen tissues can be minced or homogenized and then the resulting cells can be lysed using a mixture of enzyme and detergent, see, for example, Maniatis, Molecular Cloning, a Laboratory Manual, Cold Spring Harbor Laboratory, 1982. The nucleic acids can be extracted using standard techniques such as phenol and chloroform extraction, and ethanol precipitation. As an example, melanoma tumors were scored as exhibiting LOH when one allele showed ≧50% reduction of peak intensity for tumor DNA as compared to the corresponding allele identified in the control DNA (see Example 2 below).

Moreover, the invention provides a method of predicting the possible response of a cancer patient to a therapy. The method comprises the steps of obtaining a sample from the patient and detecting one or more DNA markers in the 12q22-23 region. LOH of the markers indicates the patient is less likely to respond to a cancer therapy. As shown in Example 2 below, patients with stage IV melanoma are less responsive to the BC treatment if they are LOH positive.

It is another object of the invention to provide packaged products for diagnosing, staging and monitoring cancer patients. Such a product includes a container, one or more agents for detecting one or more DNA markers at the 12q22-23 region in a sample, and an insert associated with the container. The insert may be a label or an instruction sheet with the following information: (1) the sample contains acellular DNA; (2) the sample is from a subject suffering from a metastatic cancer, and LOH of the markers indicates a low probability of survival; or (3) the sample is from a subject suffering from cancer, and LOH of the markers indicates a low probability of responsiveness to a therapy.

In a preferred embodiment, the product may contain a set of nucleic acid probes for specified alleles for which the patient is homozygous or heterozygous to detect LOH in these specified alleles. This provides a measure of the extent of genetic change in a neoplastic tissue or a biological fluid which can be correlated with a diagnosis or prognosis. In one specific embodiment, the presence or absence of a specific allele or combination of alleles is tested by amplification of regions of the DNA markers using pairs of primers which bracket specific regions of the DNA markers on specific chromosome arms containing repeat sequences with polymorphism. Preferably, the assay uses fluorescent labeling of DNA with multiple types of chromophores. However, radioactive and other labeling techniques known in the art also may be used.

The product may comprise a carrier means being compartmentalized to receive in close confinement one or more container means such as vials, tubes, and the like, each of the container means comprising one of the separate elements to be used for detecting DNA markers. Such elements include a labeled primer pair for amplifying a DNA marker. The product also may include an enzyme for reverse transcribing RNA to provide cDNA, a DNA polymerase for amplifying the target DNA, appropriate amplification buffers and deoxyribonucleoside triphosphates. The nucleic acids in the product may be provided in solution or lyophilized form. Preferably, the nucleic acids will be sterile and devoid of nucleases to maximize shelf-life.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### EXAMPLES

### Example 1

### Allelic Imbalance of 12q22-23 Associated with APAF-1 Locus Correlates with Poor Disease Outcome in Cutaneous Melanoma

Methods and Materials

Tumor DNA collection and preparation. Primary (n=62) and metastatic melanoma (n=112) were collected from 164 patients including 10 cases which we collected paired primary and metastatic tumors. Institutional Review Board approval and histopathologic confirmation from Saint John's Health Center and John Wayne Cancer Institute joint committee were obtained prior to study initiation. Tumor tissues were reviewed by the pathologist to confirm histopathologic status. Melanoma tissue sections were cut at 5 μm thickness and stained with hematoxylin for microdissection. Tumor cells were collected using the PixCell II Laser Capture Microdissection (LCM) System (Arcturus Engineering, Mountain View, Calif.) as previously described (Hoon et al., 2002, Methods Enzymol. 356:302-309). Captured cells were digested with proteinase K at 50° C. overnight, followed by heat denaturation at 95° C. for 10 min. Lysate was directly used for PCR as previously described (Hoon et al., 2002, Methods Enzymol. 356:302-309; and Nakayama et al., 2001, Am. J. Pathol. 158:1371-1378). Control (non-tumor) DNA for each melanoma patient was obtained from their peripheral blood lymphocytes when available, or microdissected from tumor-adjacent normal tissue as previously described (Nakayama et al., 2001, Am. J. Pathol. 158:1371-1378).

Microsatellite analysis. LOH was assessed using four microsatellite markers (D12S1657, D12S393, D12S1706, D12S346) encompassing the APAF-1 gene locus (12q22-23). For primary melanoma, microsatellite marker D9S157, one of the most frequent LOH markers in cutaneous melanoma, was also examined as a control marker. PCR primer sets for specific allele loci were obtained from Research Genetics, Inc. (Huntsville, Ala.). Forward primers were labeled with WellRed phosphoramidite-linked dye or active ester-labeled dye. The PCR amplification was performed in a 10-ul reaction volume with 1-ul template for 40 cycles of 30 s at 94° C., 30 s at 55° C., and 30 s at 72° C., followed by a 7 min final extension at 72° C. PCR product separation was performed using capillary array electrophoresis (CAE CEQ 8000XL, Beckman Coulter, Inc., Fullerton, Calif.). Peak signal intensity and relative size were generated by a fragment analysis system software (Beckman Coulter). Tumors were scored as exhibiting LOH when one allele showed ≧50% reduction of peak intensity for tumor DNA as compared to the corresponding allele identified in the control DNA. The markers showing homozygosity, microsatellite instabilities, and insufficient PCR amplification were scored as non-informative. We considered a specimen to be APAF-1 LOH positive when LOH is found for any of the four markers assessed and considered specimens to be APAF-1 LOH negative if they demonstrated retention of allele closer to APAF-1 locus than the marker that is found LOH positive. Eight primary melanomas and 12 metastatic melanomas were excluded from APAF-1 LOH evaluation because fewer than two markers was informative. In cases of doubtful LOH interpretation, sample assays were repeated to verify and confirm the results.

RT-PCR assay. For APAF-1 mRNA expression analysis, one to five 5 um thick Hematoxilin Eosin-stained sections were prepared from 22 paraffin-embedded melanoma tumors (1 primary melanoma and 21 metastatic melanomas). Tumor tissues were microdissected using LCM, RNA was extracted using a modified protocol of the Paraffin Block RNA Isolation Kit (Ambion, Austin, Tex.), total RNA was quantified (Takeuchi et al., 2003, Cancer Res. 63:441-448). Reverse-transcriptase reactions were performed using Moloney murine leukemia virus reverse-transcriptase (Promega, Madison, Wis.) with oligo-dT and random hexamer primers, as previously described (Bostick et al., 1999, J. Clin. Oncol. 17:3238-3244). For all specimen analysis, the PCR reaction mixture contained cDNA template from 250 ng of total RNA: 1 uM of APAF-1 F primer 5′-ACATTTCTCACGATGCTACC-3′ (SEQ ID NO:1); 1 uM of APAF-1 R primer 5′-CAATTCATGAAGTGGCAA-3′ (SEQ ID NO:2); and 0.3 uM FRET probe 5′-FAM-TGCTGACAAGACTGCAAAGATCTG-BHQ-1-3′ (SEQ ID NO:3). Positive controls used in all assays were paraffin-embedded normal lymph nodes and melanoma cell lines. Negative control was all PCR reagents with no template. The house-keeping gene GAPDH was used as an internal reference gene to determine the integrity of RNA and the data collected was sequentially used to normalize APAF-1 mRNA expression level. Quantitative RT-PCR assay was performed on the iCycer iQ RealTime thermocycler detection system (Bio-Rad Laboratories, Hercules, Calif.) (Takeuchi et al., 2003, Cancer Res. 63:441-448). The standard curve was established for quantifying mRNA copy numbers by using nine known copy numbers of serial diluted (10⁰ to 10⁸ copies) plasmids containing APAF-1 and GAPDH cDNA, respectively. Copy numbers of APAF-1 and GAPDH mRNA were established by the respective standard curve. APAF-1 mRNA level was determined by APAF-1:GAPDH mRNA log ratio (Takeuchi et al., 2003, Cancer Res. 63:441-448).

APAF-1 promoer region methylation analysis. Methylation of APAF-1 promoter region was assessed in 19 of 22 samples that we analyzed for APAF-1 mRNA expression and an additional 30 metastatic melanomas. The assay involved sodium bisulfite modification followed by methylation-specific PCR (MSP) to determine the methylation status of APAF-1 promotor region as previously described (Spugnardi et al., 2003, Cancer Res. 63:1639-1643). As a positive and negative control, SssI methylase treated and untreated normal DNA was used, respectively. Sodium bisulfite modification was performed as previously reported (Olek et al., 1996, Nucleic Acids Res. 24:5064-5066). MSP was performed using fluorescently labeled methylation and unmethylation-specific primers. Primers used for amplification were as follows: methylated APAF-1 F primer 5′-GTCGTTGTTCGAGTTCGGTA-3′ (SEQ ID NO:4), R primer 5′-GCGTAAAAATACCCGCCTAC-3′ (SEQ ID NO:5); unmethylated APAF-1 F primer 5′-GGGTGTGTTGTTGTTGTTTGA-3′ (SEQ ID NO:6) and R primer 5′-AAATACCCACCTACCCCACA-3′ (SEQ ID NO:7). Detection of PCR products was analyzed by capillary array electrophoresis as described in microsatellite analysis.

Statistical analysis. The relation between APAF-1 LOH and other variables were assessed using Fisher's exact test. To investigate the association between APAF-1 LOH and APAF-1 mRNA expression, Student's t test was used. Survival was determined from the date of melanoma surgery to death or last follow-up. Survival curves were assessed by the Kaplan-Meier method and differences between curves were analyzed using the log-rank test. Cox's proportional hazard regression models were used for multivariate and univariate analyses and for calculation of the risk-ratio (Hoon et al., 2000, Cancer Res. 60:2253-2257). Stepwise variable selection was adopted with a selection rule of P<0.1 for variables.

Results

LOH frequency in primary melanomas. In the analysis of 62 primary melanomas, the frequencies of LOH for each microsatellite marker in informative cases were 20%, 31%, 13%, 17%, and 47% at D12S1657, D12S393, D12S1706, D12S346, and D9S157, respectively (Table 1).

TABLE 1
Frequency of LOH of microsatellite markers at 12q22-23

| Melanoma | | | | | | |
|---|---|---|---|---|---|---|
| specimen | D12S1657 | D12S393 | D12S1706 | D12S346 | 12q22-23 | D9S157 |
| Primary | 20% (8/38) | 31% (11/36) | 13% (7/54) | 17% (8/47) | 31% (19/62)^{a} | 47% (27/58) |
| Metastasis | 23% (14/61) | 35% (23/66) | 17% (16/93) | 21% (19/90) | 41% (46/109)^{a} | NE |

(number of LOH/number of informative)
^{a}(number of cases with at least one marker LOH/number of cases with at least one marker informative)
NE, not examined

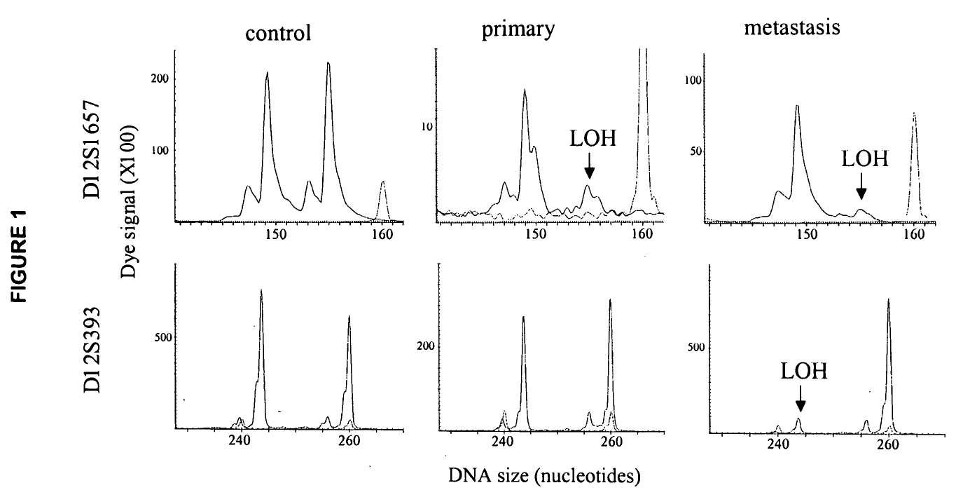

D9S157, one of the most frequent microsatellite markers with LOH found in primary cutaneous melanomas, was used as a control marker for assay efficiency. Allelic imbalance of this control marker (D9S157) was detected in 3 of 10 (30%) thin (≦1.0 mm ) primary melanomas. Representative results are shown in FIG. 1. APAF-1 LOH was identified in 10 of 54 primary melanomas (17%) (Table 2) by the defined criteria outlined in the Materials and Methods.

TABLE 2
Characteristics of primary melanoma patients

| Characteristics | | n | LOH/informative cases |
|---|---|---|---|
| Total patients | | 62 | 10/54 (19%) |
| Sex | male | 41 | 6/35 (17%) |
| | female | 21 | 4/19 (21%) |
| Age | <50 | 16 | 3/14 (21%) |
| | ≧50 | 46 | 7/40 (18%) |
| Breslow | ≦1.0 | 9 | 0/8 (0%) |
| thickness | 1.01-2.0 | 16 | 2/14 (14%) |
| | 2.01-4.0 | 20 | 4/16 (25%) |
| | >4.0 | 15 | 3/14 (21%) |
| | unknown | 2 | 1/2 (50%) |
| Site | head | 17 | 4/14 (29%) |
| | trunk | 15 | 0/14 (0%) |
| | extremities | 15 | 4/14 (29%) |
| | hand & foot | 15 | 2/12 (17%) |
| AJCC Stage | I | 18 | 1/18 (6%) |
| | II | 21 | 6/18 (33%) |
| | III | 16 | 1/11 (9%) |
| | IV | 2 | 0/2 (0%) |
| | unknown | 5 | 2/5 (40%) |

When stratified according to the primary tumor Breslow thickness, the frequency of APAF-1 LOH in primary melanomas of <1.0-mm, 1.01-2.0-mm, 2.01-4.0-mm, and >4.0-mm was 0% (0 of 8), 14% (2 of 14), 25% (4 of 16), and 21% (3 of 14), respectively. Breslow thickness data was not available in two patients. There was no significant pattern of APAF-1 LOH related to any particular Breslow thickness as further evidenced by the lack of significance in APAF-1 LOH frequency between ≦1.0-mm and >1.0-mm melanomas or between <2.0-mm and >2.0-mm melanomas. Age, sex, and site showed no significant correlation with APAF-1 LOH in primary melanomas.

LOH frequency in metastatic melanomas. In the analysis of 112 metastatic melanomas, the frequency of LOH for each microsatellite marker in informative cases was 23%, 35%, 17%, and 21% at D12S1657, D12S393, D12S1706, and D12S346, respectively (Table 1). APAF-1 LOH was found in 36 of 98 metastatic melanoma patients (37%) by the defined criteria in the Material and Methods (Table 3).

TABLE 3
Characteristics of metastatic melanoma patients

| | | | | LOH/informative |
|---|---|---|---|---|
| Characteristics | | | n | cases |
| Total patients | | | 112 | 36/98 (37%) |
| Sex | | Male | 77 | 21/70 (30%) |
| | | Female | 35 | 15/28 (54%) |
| Age | | <50 | 50 | 13/45 (29%) |
| | | ≧50 | 61 | 23/52 (44%) |
| | | unknown | 1 | 0/1 (0%) |
| AJCC Stage | III | | 83 | 26/72 (36%) |
| | | RLM | 44 | 11/40 (28%) |
| | | ITM | 39 | 15/32 (47%) |
| | IV | | 29 | 10/26 (38%) |

| | | | |
|---|---|---|---|
| | lung | 9 | 3/9 (33%) |
| | bowel | 12 | 2/10 (20%) |
| | liver | 1 | 1/1 (100%) |
| | other sites | 7 | 4/6 (67%) |
| Breslow thickness | <=1.0 | 13 | 2/10 (20%) |
| (primary tumor)^{b} | 1.01-2.0 | 21 | 5/18 (28%) |
| | 2.01-4.0 | 24 | 8/19 (42%) |
| | >4.0 | 8 | 5/8 (63%) |
| | unknown | 17 | 6/17 (35%) |

^{b}Available for AJCC Stage III melanoma

The frequency of allelic imbalance was significantly higher in metastatic melanomas than in primary melanomas (P=0.02), but there was no significant difference in the frequency of allelic imbalance associated with American Joint Committee on Cancer (AJCC) stage III (36%) versus stage IV (38%) melanoma patients. We then stratified the AJCC stage III patients into patients with RLM (n=44) or ITM (n=39) because of their known pathologic and clinical outcome differences. Although both RLM and ITM are classified as AJCC stage III disease, their outcomes are vastly different; ITM have an unusual propensity to recur rapidly and frequently after excision of the lesions (Nakayama et al., 2001, Am. J. Pathol. 158:1371-1378). In our analysis, ITM demonstrated a trend toward more frequent APAF-1 LOH than RLM, although this difference was not significant (P=0.09).

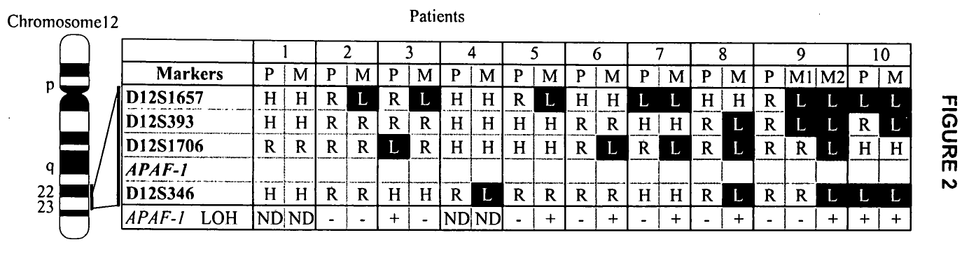

Comparison between paired primary and metastatic tumors. To further assess whether APAF-1 was associated with tumor progression, we assessed 10 paired primary and metastatic tumors. Frequency of allelic imbalance at the APAF-1 locus was 70% in metastatic lesions versus 20% in primary tumors (FIG. 2, P: primary melanoma; M: metastatic melanoma; R: retention of heterozygosity; L: LOH; H: homozygous; and ND: not determined). Only one patient showed LOH in the primary tumor which was not detected in the paired metastatic lesion. This finding may be due in part to primary tumor heterogeneity or it may involve a different tumor clone from the primary lesion that produced the metastasis. Nevertheless, the finding of more prevalent loss of APAF-1 gene loci in metastases compared to primary tumors suggests a role in tumor progression.

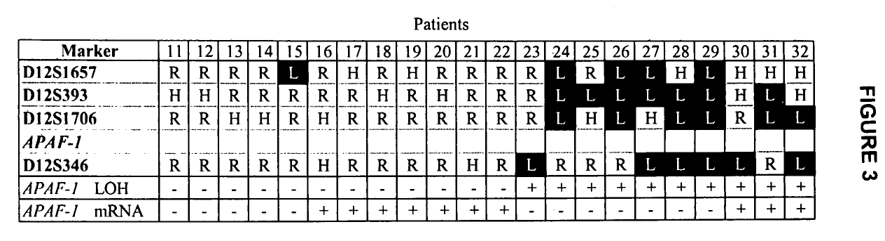

APAF-1 mRNA expression. Twenty-two melanomas (one primary and 21 metastatic) were assessed for correlation of APAF-1 mRNA expression and LOH in chromosome 12q22-23. APAF-1 mRNA expression level was normalized with GAPDH mRNA. APAF-1 mRNA expression level were significantly different between APAF-1 LOH positive and negative tumors (Student's t test P=0.030). Seven of 10 (70%) tumors with APAF-1 LOH had decreased APAF-1 mRNA level (APAF-1:GAPDH log ratio <0.1), whereas 5 of 12 (42%) tumors that demonstrated APAF-1 gene retention decreased APAF-1 mRNA level. Referring to FIG. 3, APAF-1:GAPDH log ratio was used to determine the loss of APAF-1 mRNA level. R: retention of heterozygosity; L: loss of heterozygosity; and H: homozygous. Our work supports previous work (Soengas et al., 2001, Nature 409:207-211) indicating that APAF-1 LOH decreased APAF-1 mRNA expression. This observation demonstrated a haploinsufficiency effect of LOH of APAF-1 locus. We assessed APAF-1 promoter methylation by MSP. No methylation of APAF-1 promotor region was found in all 49 tumor specimens assessed.

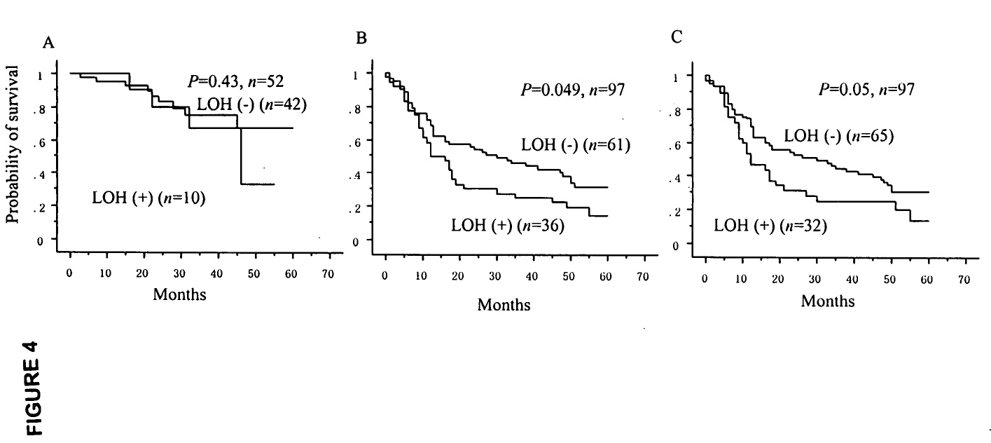

APAF-1 LOH correlation with survival. To further determine whether the identification of APAF-1 loss in melanoma relates to tumor progression and affects disease outcome, APAF-1 locus imbalance in relation to disease outcome was analyzed. Fifty-two primary and 97 metastatic melanomas were assessed in patients with clinical follow-up data. In patients with primary melanoma, there was no correlation between APAF-1 status and overall survival at a mean follow-up of 39 mos (log-rank test; P=0.43) (FIG. 4A). In contrast, in patients with AJCC stage III/IV melanoma, the presence of APAF-1 LOH in their metastatic tumor was significantly associated with a decreased overall survival at a mean follow-up of 27 mos (log-rank test; P=0.049) (FIG. 4B). Interestingly, when we applied the APAF-1 LOH definition for the previously located APAF-1 locus between D12S1657 and D12S393, allelic imbalance in that region also significantly correlated with a decreased overall survival in AJCC stage III/IV patients (log-rank test; P=0.05; FIG. 4C). Both sets of Kaplan-Meier curves for AJCC stage III/IV melanoma (FIG. 4B and FIG. 4C) show a significant correlation between presence of the genetic aberration and decreased survival.

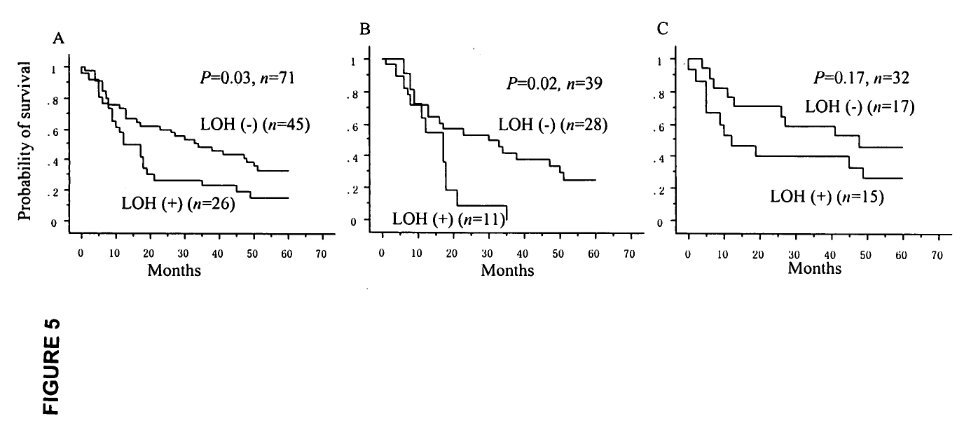

FIG. 5 shows correlation between survival and APAF-1 LOH in AJCC stage III melanoma (A), AJCC stage III melanoma with RLM (B), and AJCC stage III melanoma with ITM (C). Kaplan-Meier survival curves (FIG. 5A and FIG. 5B) demonstrated that APAF-1 LOH (+) group had a significantly poorer overall survival compared with the APAF-1 LOH (-) group. The difference in overall survival of patients with APAF-1 LOH in their metastatic melanoma was more apparent in AJCC stage III (FIG. 5A) than stage IV melanoma (log-rank test; P=0.03, P=0.81, respectively). AJCC stage III melanomas were further categorized into RLM and ITM, because each type of regional metastasis has a distinct pathology and clinical outcome. APAF-1 LOH in RLM had a significantly worse survival outcome (log-rank test; P=0.02) compared to APAF-1 LOH in ITM (log-rank test; P=0.17) (FIG. 5B and FIG. 5C). Cox's proportional hazard models for stage III metastatic tumors showed that APAF-1 LOH had a significant effect on overall survival (risk ratio 1.35, 95% confidence interval 1.02-1.79, P=0.04) in univariate analysis. For multivariate analysis, only the AJCC stage III metastatic pattern (RLM versus ITM) and APAF-1 LOH were chosen as variables by stepwise variable selection; RLM versus ITM, risk ratio 0.76, 95% confidence interval 0.57-1.02, P=0.07; and APAF-1 LOH, risk ratio 1.44, 95% confidence interval 1.08-1.93, P=0.01.

Discussion

We demonstrated a high frequency of LOH at 12q22-23 locus in primary and metastatic melanomas. For metastatic melanoma, the frequency was similar to that reported by Soengas et al. (Nature 409:207-211, 2001). However, we demonstrated that the frequency of APAF-1 LOH was significantly lower in primary melanomas than in metastatic melanoma. Among 10 paired primary and metastatic tumors, LOH at the APAF-1 locus was more frequent in metastatic tumors than primary tumors. Furthermore, loss of APAF-1 was a more significant factor for progression than initiation of melanoma. The allelic imbalances at the APAF-1 locus, associated to disease progression, may be the result of genetic alterations accumulated through a prolonged period of chromosomal instability during melanoma progression.

Previous LOH studies in melanoma have shown allelic imbalances on chromosome loci 1p, 3p, 6q, 10q, and 11q, with the most frequent events occurring at 9p21 ranging from 30˜50% (Healy et al., 1995, Genes Chromosomes Cancer 12:152-156; Healy et al., 1998, Oncogene 16:2213-2218; Walker et al., 1994, Int. J. Cancer 58:203-206; and Fujimoto et al., 1999, Oncogene 18:2527-2532). Chromosome 12q22-23 should now be considered to have a significant allelic imbalance and is comparable to the frequency of other allelic chromosomal imbalances reported for cutaneous melanoma. Clinicopathological correlations have shown that LOH on 9p and 10q are early events during melanoma progression, followed by LOH on 1p, 6q, and 11q (Morita et al., 1998, J. Invest. Dermatol. 111:919-924; and Takata et al., 2000, Int. J. Cancer 85:492-497). LOH on 10q in primary melanoma has been correlated to poor prognosis, and LOH on 6q has been correlated with metastasis (Healy et al., 1998, Oncogene 16:2213-2218; Millikin et al., 1991, Cancer Res. 51:5449-5453; and Shirasaki et al., 2001, Cancer Res. 61:7422-7425). These studies need further validation by larger sample sizes. Although allelic imbalance is frequent on various chromosome regions in melanoma, specific genes for many regions have yet to be identified. Most of the analyses of allelic imbalance in cutaneous melanomas have been performed on metastatic tumors. Very limited studies on large sample sizes have been reported in primary melanomas of different thickness. Our analysis is one of the largest for any individual microsatellite region marker for primary melanomas.

The reduction of mRNA in tumors with LOH of APAF-1 locus demonstrated haploinsufficiency. We do not know what is the critical level of APAF-1 mRNA that relates to its functional activity at this time. We found that some cases expressed APAF-1 mRNA at lower level despite the absence of APAF-1 LOH. There may be other inactivating mechanisms of APAF-1. One possible mechanism is methylation of APAF-1. We also analyzed APAF-1 promoter region by sodium bisulfite modification-based MSP assay and did not detect hypermethylation in the APAF-1 promotor region. Soengas et al. (Nature 409:207-211, 2001) also examined hypermethylation on CpG islands in the APAF-1 5′-untranslated region, but no extensive methylation was found in this region. Interestingly, they showed reactivation of APAF-1 by treating cultured melanoma cells with the methylation inhibitor (5-aza-2′-deoxycytidine) or histone deacetylase inhibitor (Tricostatin A). This indicates that APAF-1 mRNA expression may be also controlled by a promoter region further upstream or by a transcription regulating factor(s).

In a previous study, APAF-1 gene was thought to be located between D12S1657 and D12S393 (Soengas et al., 2001, Nature 409:207-211), but the current genome update of the NCBI database indicates that APAF-1 gene is located between D12S1706 and D12S346, which is more distal to the centromere on chromosome 12q. This designation change of >0.3 Mb indicates that the 42% rate of APAF-1 LOH reported by Soengas et al. would decrease to 33%. In our study, the frequency of LOH for each marker was relatively higher in D12S1657 and D12S393 than in D12S1706 and D12S346. Survival curve analysis showed a significant difference if APAF-1 LOH was defined to be between D12S1657 and D12S393. The studies strongly suggest the likelihood of another tumor suppressor gene or tumor-related gene in the vicinity of microsatellite markers D12S1657 and D12S393. Further detailed analysis is needed to identify any potential gene(s) in this region that may influence melanoma progression.

One problem in analyzing LOH is homozygous deletion of the locus of interest. It is difficult to detect homozygous deletion in clinical samples using microsatellite markers, because these markers may show retention of heterozygosity due to PCR product amplification from normal cell contamination. According to our definition of APAF-1 LOH, it was considered negative when D12S1706, the nearest marker among markers upstream of APAF-1, showed retention, even if further marker D12S1657 or D12S393 showed LOH. In such cases, there may be homozygous deletion at D12S1706 locus. This may explain why more frequent LOH was found at D12S1657 and D12S393 than D12S1706 and D12S346.

The ability to escape from apoptosis is a critical factor for melanoma cells to survive under selective pressures such as host immune responses and physiological factors. Although melanoma cells are known to be highly immunogenic compared to other types of cancers, they can be highly resistant to host immune attacks. T-cells have been demonstrated to kill melanoma cells by granzyme-B-induced apoptosis and TRAIL-induced apoptosis. Both apoptotic mechanisms involve the mitochondrial pathway (Hersey and Zhang, 2001, Nat. Rev. Cancer 1:142-150). Loss of APAF-1 gene may play a key role in evasion from immunosurveillance and subsequently influence the response to immunotherapy. This may develop into more of an “anti-apoptosis genotype” as metastasis progress. The allelic imbalance of 12q22-23 including the loss of APAF-1 gene appears to be a major facilitator of metastasis.

It is well known that in AJCC stage III/IV melanoma the optimal treatment is surgery. Chemo-, immuno- and radiotherapy to date have not consistently or significantly improved survival by any substantial levels over the last decade. In our study, the significant association between APAF-1 LOH and the survival of patients with stage III and stage IV melanoma supports loss of APAF-1 as an important factor for establishment of metastasis. Of note, there was no correlation between APAF-1 loss or 12q22-23 allelic imbalance and Breslow thickness of the primary tumor. Clinically, increasing Breslow thickness of the primary tumor is significantly associated with worse disease outcomes. This suggests that APAF-1 is not a key factor in vertical growth phase progression in melanomas. More importantly, this suggests that 12q22-23 allelic imbalance or APAF-1 loss are linked to the progression of metastasis rather than the initiation of melanoma.

We have demonstrated the subsequent progressive loss of APAF-1 during different defined stages of melanoma development from primary tumor to systemic metastasis. Our results suggest that APAF-1 gene loss is important for the progression of cutaneous melanoma and becomes a dominant functional genotypic aberration with advancing stage of disease. This was clearly demonstrated in the comparison of primary and metastatic melanomas. If metastatic melanomas are more likely to survive through inactivation of the APAF-1 intrinsic apoptotic pathway, development of therapeutics to supplement APAF-1 function in this pathway might improve treatment efficiency (Satyamoorthy et al., 2001, Trends Mol. Med. 7:191-194). This APAF-1 gene loss may be used as a potential prognostic marker of metastatic melanoma, and it may indicate likelihood of response to various therapies. Future studies on prospective frozen melanoma tissues may allow validation of the role of this gene loss in melanoma patient disease outcome.

We conclude that LOH at the 12q22-23 region is a significant genetic alteration in melanoma, which may harbor more than one tumor-related gene. The study strongly suggests that APAF-1 gene loss as a clinicopathological factor facilitating melanoma metastasis. Further studies are needed to determine if this regional allelic imbalance contributes to resistance to therapy. If patients with metastatic tumors having 12q22-23 allelic imbalance are unlikely to respond to chemo- or immunotherapy, this observation may be useful as a stratification factor in future studies. We are entering an era of molecular targeted therapies that are better tailored to specific tumor subsets. Concomitant to this progress, we must have in place reliable determination of in vivo tumor susceptibility to the therapy with the appropriate targeted killing mechanisms such as inactivation of the apoptosis pathway(s).

### Example 2

### Allelic Imbalance on 12q22-23 in Serum DNA of Melanoma Patients Predicts Disease Outcome

Materials and Methods

Serum DNA collection and preparation. Forty-nine AJCC stage IV melanoma patients treated with concurrent BC regimen of dacarbazine (DTIC), cisplatin, vinblastin, interferon α-2b, IL-2, and tamoxifen as previously reported (O'Day et al., 1999, J. Clin. Oncol. 17:2752-2761; and O'Day et al., 2002, Clin. Cancer Res. 8:2775-2781) were selected (Table 4).

TABLE 4
Clinical characteristics of BC patients

| | | | # AI/# informative cases | |
|---|---|---|---|---|
| Characteristics | | n | pre-BC serum | post-BC serum |
| Total patients | | 49 | 16/44 (36%) | 16/44 (36%) |
| Sex | male | 38 | 14/35 (40%) | 13/35 (37%) |
| | female | 11 | 2/9 (22%) | 3/9 (33%) |
| Age (median 45) | <50 | 33 | 12/30 (40%) | 10/30 (33%) |
| | ≧50 | 16 | 4/14 (29%) | 6/14 (43%) |
| BC response | | | | |
| Responder | CR | 13 | 1/12 (8%) | 4/12 (33%) |
| | PR | 10 | 3/9 (33%) | 4/9 (44%) |
| | SD | 3 | 1/3 (33%) | 1/3 (33%) |
| Non-responder | PD | 23 | 11/20 (55%) | 7/20 (35%) |
| LDH | ≦190 | 22 | 7/19 (37%) | 6/19 (32%) |
| | >190 | 27 | 9/25 (36%) | 10/25 (40%) |
| # of metastasis sites | ≦2 | 28 | 10/25 (40%) | 7/25 (28%) |
| | >2 | 21 | 6/19 (32%) | 9/19 (47%) |

Institutional Review Board approval and histopathologic confirmation from Saint John's Health Center and John Wayne Cancer Institute joint committee were obtained prior to study initiation. Blood was drawn for serum prior to administration of BC (pre-BC serum) and after completion of BC cycles (post-BC serum). Patients were divided into two groups (responders and non-responders) based on response criteria developed by the Response Evaluation Criteria in Solid Tumors Group (Therasse et al., 2000, J. Natl. Cancer Inst. 92:205-216). Patients who showed complete response (CR) (n=13), partial response (PR) (n=10) or stable disease (SD) (n=3) were included in the responder group (n=26), whereas patients demonstrating progressive disease were deemed non-responders (n=23). Median completed cycles of BC were six for responder group and three for non-responder group.

Ten ml of blood was collected in red top separator serum tubes (Becton Dickinson, Franklin Lakes, N.J.), serum was immediately separated from cells by centrifugation (3000 rpm, 15 min), and filtered through a 13-mm serum filter (Fisher Scientific, Pittsburgh, Pa.). Serum was aliquoted and cryopreserved at −30° C. DNA was extracted from 800 ul of serum using QIAamp extraction kit (Qiagen, Valencia, Calif.) as previously described (Taback et al., 2001, Cancer Res. 61:5723-5726). Control DNA for each melanoma patients was obtained from the respective peripheral blood lymphocytes.

Microsatellite analysis. Four microsatellite markers (D12S1657, D12S393, D12S1706, D12S346) encompassing the APAF-1 gene locus (12q22-23), which were also used in previous tumor study (Fujimoto et al., 2004, Cancer Res.), were used for this analysis. The locations of microsatellite markers and APAF-1 gene were checked using the National Center for Biotechnology Information database. PCR primer sets for specific allele loci were obtained from Research Genetics, Inc. (Huntsville, Ala.). Forward primers were labeled with WellRed phosphoramidite-linked dye or active ester-labeled dye. The PCR amplification was performed in a 10-ul reaction volume with 1-ul template for 40 cycles of 30 s at 94° C., 30 s at 55° C., and 30 s at 72° C., followed by a 7 min final extension at 72° C. PCR product separation was performed using capillary array electrophoresis (CAE CEQ 8000×L, Beckman Coulter, Inc., Fullerton, Calif.). Peak signal intensity and relative size were generated by a fragment analysis system software (Beckman Coulter). AI were defined when one allele showed ≧40% reduction of peak intensity for serum DNA as compared to the corresponding allele identified in the control DNA. The markers showing homozygosity, microsatellite instabilities, and insufficient PCR amplification were scored as non-informative. Five serums in which ≦1 marker was informative were excluded from clinical correlation analysis because of difficulty to define AI status on this locus by one or less marker. All AI were confirmed by repeating the assay.

Statistical analysis. Correlation between AI on 12q22-23 and BC response was assessed using Fisher's exact test. Survival length was determined from the first day of BC treatment, to death or the date of last follow-up. Survival curves were drawn by Kaplan-Meier method and differences between curves were analyzed using the log-rank test. Cox's proportional hazards regression model were used for multivariate analysis and calculation of the risk ratio (Hoon et al., 2000, Cancer Res. 60:2253-2257). Stepwise variable selection was adopted with a selection rule of P<0.1 for variables.

Results

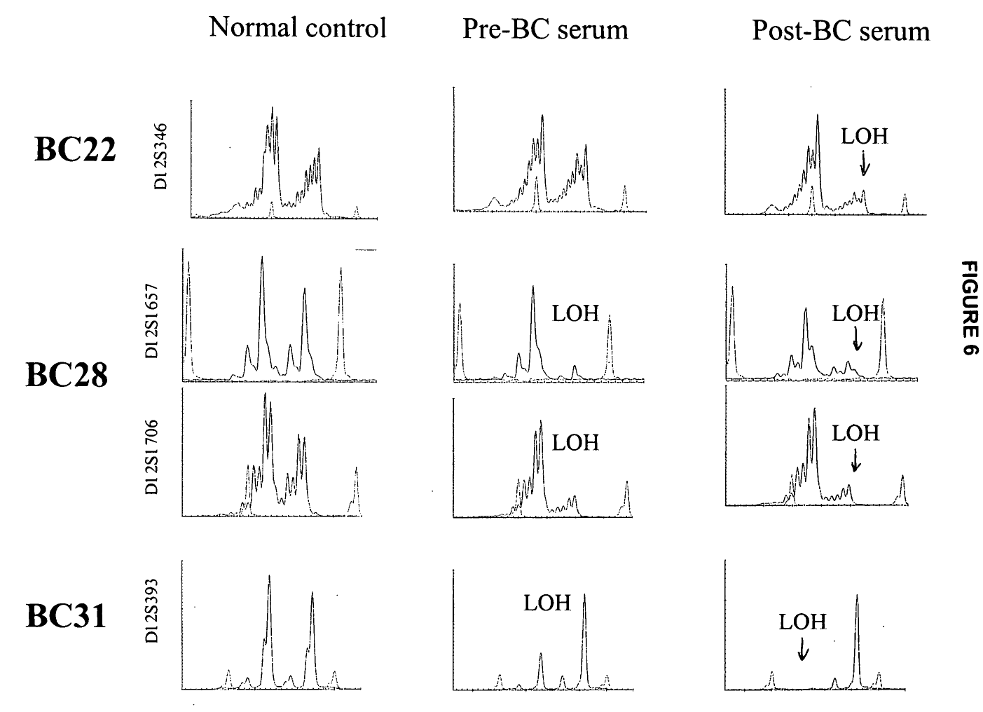

Frequency of AI on 12q22-23. In the analysis of all 49 patients serums, the frequencies of AI for each microsatellite marker in informative cases were 22% (6 of 27), 15% (5 of 34), 11% (4 of 38), and 20% (8 of 41) in pre-BC serum and 19% (5 of 26), 22% (7of 32), 13% (5 of 38), and 17% (7 of 41), in post-BC serum at D12S1657, D12S393, D12S1706, and D12S346, respectively (FIG. 6 and Table 5). FIG. 6 shows representative capillary array electrophoresis results of 3 cases demonstrating AI in pre-BC and post-BC serum. Arrows indicate decreased peak showing AI.

TABLE 5
Correlation with BC responses

| | | | BC response | P value |
|---|---|---|---|---|
| Characteristics | | n | Responder | on-responder |
| Total patients | | 49 | 26 | 23 |
| Pre-BC serum | R | 28 | 19 | 9 |
| | 0.0285 | | | |
| | AI | 16 | 5 | 11 |
| | ND | 5 | 2 | 3 |
| Post-BC serum | R | 28 | 15 | 13 |
| | 0.999 | | | |
| | AI | 16 | 9 | 7 |
| | ND | 5 | 2 | 3 |
| Sex | male | 38 | 21 | 17 |
| | 0.734 | | | |
| | Female | 11 | 5 | 6 |
| Age (median 45) | <50 | 33 | 15 | 18 |
| | 0.143 | | | |
| | ≧50 | 16 | 11 | 15 |
| LDH | ≦190 | 22 | 14 | 8 |
| | 0.252 | | | |
| | >190 | 27 | 12 | 15 |
| # of metastasis sites | ≦2 | 28 | 17 | 11 |
| | 0.257 | | | |
| | >2 | 21 | 9 | 12 |

R: retention of 12q22-23 locus;
ND: not determined;
LDH: lactate dehydrogenase.

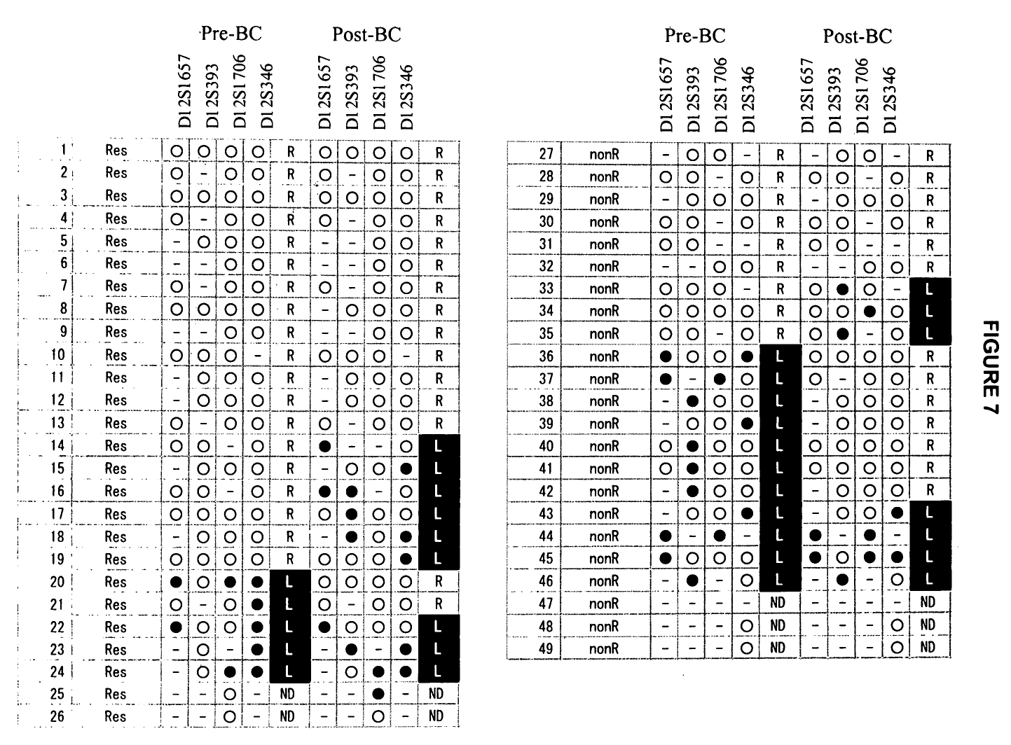

Five patient serums in which ≦1 marker was informative were excluded from clinical correlation analysis because of the difficulty to define AI status on this locus from one or fewer informative marker. Cases with AI positive in at least one marker was found in 16 of 44 (36%) pre-BC serum, and 16 of 44 (36%) post-BC serum. FIG. 7 shows results of AI on 12q22-23 for all sera. Res: responder; NonR: non responder; ∘: retention of heterozygosity; ●: AI; -: non-informative; L: allelic loss at 12q22-23; R: allele retained at 12q22-23; and ND: allele status not determined.

Correlation to clinical outcome. Loss of APAF-1 gene may account for cellular resistance to chemotherapy. AI on 12q22-23 status in pre-BC serum was assessed to predict patients likely to respond to BC. The frequency of AI on 12q22-23 in pre-BC serum was significantly lower in the responder group (5 of 24, 21%) than in the non-responder group (11 of 20, 55%) (Fisher's exact test; P=0.029). There was no significant difference of the frequency of AI on 12q22-23 in post-BC serum between the responder group (9 of 24, 38%) and the non-responder group (7 of 20, 35%). No other known prognostic factor associated with BC response (Table 5).

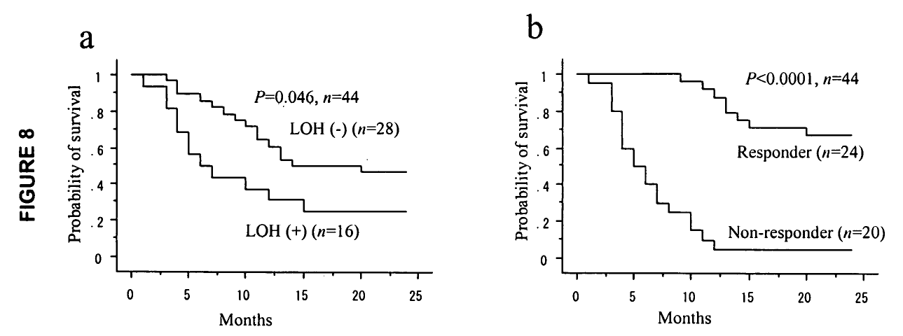

AI positive group in pre-BC serum had significantly worse survival than the AI negative group (log-rank test P=0.046; FIG. 8a). Response to BC had significant effect on survival (log-rank test P<0.0001; FIG. 8b). Using a Cox's proportional hazards regression model, AI in pre-BC serum and elevated lactate dehydrogenase (LDH) (>190 IU/liter) significantly correlated with survival (AI in pre-BC serum, risk ratio 2.33, 95% confidence interval 1.08-5.03, P=0.032; LDH, risk ratio 2.82, 95% confidence interval 1.23-6.54, P=0.0 15). Other prognostic factors in the model such as sex, age, and number of metastatic disease sites were not significant. Due to the significant correlation of AI with BC response, BC response was excluded from variables.

LOH of APAF-1 in other cancers. APAF1 loss has been associated with other cancers such as colon cancer and breast cancer (Table 6 and Table 7).

TABLE 6
LOH of APAF-1 in colon cancer

| | LOH | Retention | Total | % LOH |
|---|---|---|---|---|
| Adenomas | 0 | 33 | 33 | 0% |
| Primary cancers | 9 | 33 | 42 | 21% |
| Liver metastases | 15 | 13 | 28 | 54% |

TABLE 7
LOH of APAF-1 in breast cancer

| | LOH | Retention | Total | % LOH |
|---|---|---|---|---|
| Primary cancers | 7 | 21 | 28 | 25% |

Therefore, it is possible to use APAF-1 loss as a serum and tissue marker for diagnosis and monitoring in these cancers.

Discussion

Since the discovery of circulating tumor-derived DNA in serum/plasma, investigators have sought to determine the clinical utility of serum DNA of cancer patients. We focused on AJCC stage IV melanoma patients and assessed the clinical utility of microsatellite analysis of circulating serum DNA as a predictive marker of BC response. Tumor cells susceptibility to undergo apoptosis may be an important determining factor for BC response in melanoma patients. Soengas et al. demonstrated that AI on the APAF-1 gene locus was frequent and indicated that loss of APAF-1 was a major factor of chemoresistance of melanoma (Soengas et al., 2001, Nature 409:207-211). We recently demonstrated that 12q22-23 AI of metastatic melanoma tumors was associated with poorer disease outcome. The study also demonstrated that APAF-1 loss increased during tumor progression from primary to metastatic tumors (Fujimoto et al., 2004, Cancer Res.). In the present study, we demonstrated that 12q22-23 AI in serum was associated with response to BC. Our results provide the significance of APAF-1 loss as the surrogate in the immuno-chemoresistance of melanoma. A major problem of assessing treatment of systemic therapy is assessment of tumor responses. Current imaging approaches are highly subjective and provide limited information. Most importantly, one cannot perform tumor sampling to assess genetic changes. In this study, we demonstrated a new approach of assessing a tumor genetic marker associated with apoptosis.

Six responder patients with AI negative in pre-BC serum, turned into AI positive in post-BC serum. One possibility was the increasing tumor derived-serum DNA due to BC induced-apoptosis. But, recent reports measuring nucleosomes by ELISA (Trejo-Becerril et al., 2003, Int. J. Cancer. 104:663-668) or measuring fetal DNA from maternal plasma (Lo et al., 1999, Am. J. Hum. Genet. 64:218-224) indicated that circulating serum/plasma DNA was cleared rapidly and that the estimated half-life was less than 1 hour. In our study, post-BC serums were collected after completion of BC. So, post-BC serum DNA was not likely to be influenced by immediate BC-induced apoptosis of melanoma cells. BC may have induced the clonal selection of specific melanoma cells. In responder cases, BC therapy could kill APAF-1 expressing tumor cells as indicated for the majority pretreatment serum genotype in serum. Long-term BC therapy and other systemic therapies may promote selection of APAF-1 (−) clones that become eventually dominant in the metastasis. This may explain why long-term remissions are rare, and why melanoma patients with systemic metastasis are generally poor and unresponsive to chemotherapy and radiotherapy.

One of the major problems in assessing tumor genetic markers is the availability of melanoma tumor specimen from distant metastasis. The ability to assess blood for tumor genetic markers provides a novel approach to monitor tumor progression or response to therapy. Previously, we identified circulating tumor microsatellites with AI in the acellular plasma of patients with melanoma (Fujiwara et al., 1999, Cancer Res. 59:1567-1571; Nakayama et al., 2000, Ann. N. Y. Acad. Sci. 906:87-98; and Taback et al., 2001, Cancer Res. 61:5723-5626). The blood AI correlated with genetic alterations present in the respective melanoma tumors and with poorer disease outcome (Fujiwara et al., 1999, Cancer Res. 59:1567-1571). Identifying surrogate serum circulating tumor genetic determinants particularly relevant to apoptosis resistance would be of significant clinical utility for therapy stratification. Most molecular monitoring of therapeutics focus on the target gene instead of susceptibility of the tumor to be resistant to apoptosis.

Along with melanoma progression, melanoma may produce many types of clones to obtain the advantage to progress and survive. Stage IV melanoma patient tumors are often highly genetically instable and heterogenous. The genotype of serum DNA is likely to represent the genotype of the most dominant tumor clone at that time. BC may induce clonal selection whereby resistant tumor cells survive and become more dominant after systemic therapy. Therefore, it may be more efficacious to have multiple agent attacks like BC in advance stage patients.

When retention of heterozygosity in the serum DNA analysis is demonstrated, three interpretations can arise: 1) The tumor cell does not carry AI at the locus. 2) Homozygous deletion at the locus has occurred in tumor cells. 3) Tumor-derived DNA in serum can be under-detected due to small size of tumor or high normal cell derived DNA interference. These factors can affect the interpretations of results. Further refinement of the technologies and adding different markers should improve the assay efficacy.

Our results suggest that AI on 12q22-23 is an important determining factor for the response to BC, and becomes a dominant functional genotypic aberration with advancing stages of the disease. If so, then advanced melanomas are more likely to be resistant to therapy that requires the activation of the APAF-1 intrinsic apoptotic pathway. Development of therapeutics to supplement APAF-1 function in the apoptosis pathway may be needed to improve treatment efficiency in melanoma patients. This study demonstrates that detecting loss of a key apoptotic gene locus as deleted in serum can be used as a surrogate genetic determinant in cancer patients to predict the response to therapy. To our knowledge, this is the first study to evaluate the association between circulating DNA apoptosis marker on a specific gene locus and a patient's disease outcome. APAF-1 gene loss may be used as a potential prognostic marker of melanoma progression, whereby tumor assessment and serial genetic monitoring in serum can be accomplished.

While the foregoing has been described in considerable detail and in terms of preferred embodiments, these are not to be construed as limitations on the disclosure or claims to follow. Modifications and changes that are within the purview of those skilled in the art are intended to fall within the scope of the following claims. All literatures cited herein are incorporated by reference in their entirety.

1. A method of detecting DNA markers in the 12q22-23 region, comprising
providing a sample containing acellular DNA from a subject; and
detecting one or more DNA markers in the 12q22-23 region in the sample.

2. The method of claim 1, wherein the sample is a serum sample.

3. The method of claim 1, wherein the sample is a plasma sample.

4. The method of claim 1, wherein the DNA markers include D12S1657, D12S393, D12S1706, D12S346, or a combination thereof.

5. The method of claim 1, wherein the DNA markers are associated with the APAF-1 gene.

6. A method of detecting cancer, comprising
providing a sample containing acellular DNA from a subject; and
detecting one or more DNA markers in the 12q22-23 region in the sample, wherein LOH of the DNA markers is indicative of cancer.

7. The method of claim 6, wherein the sample is a serum sample.

8. The method of claim 6, wherein the sample is a plasma sample.

9. The method of claim 6, wherein the DNA markers include D12S1657, D12S393, D12S1706, D12S346, or a combination thereof.

10. The method of claim 6, wherein the DNA markers are associated with the APAF-1 gene.

11. The method of claim 6, wherein the cancer is melanoma.

12. The method of claim 11, wherein the melanoma is a primary melanoma.

13. The method of claim 11, wherein the melanoma is a metastatic melanoma.

14. The method of claim 6, wherein the cancer is colon cancer.

15. The method of claim 6, wherein the cancer is breast cancer.

16. The method of claim 6, wherein the cancer is brain cancer.

17. A method of staging cancer, comprising
providing sample containing acellular DNA from a subject suffering from cancer; and
detecting one or more DNA markers in the 12q22-23 region in the sample, wherein LOH of the DNA markers indicates a high probability of a metastatic cancer.

18. The method of claim 17, wherein the sample is a serum sample.

19. The method of claim 17, wherein the sample is a plasma sample.

20. The method of claim 17, wherein the DNA markers include D12S1657, D12S393, D12S1706, D12S346, or a combination thereof.

21. The method of claim 17, wherein the DNA markers are associated with the APAF-1 gene.

22. The method of claim 17, wherein the cancer is melanoma.

23. The method of claim 17, wherein the cancer is colon cancer.

24. The method of claim 17, wherein the cancer is breast cancer.

25. The method of claim 17, wherein the cancer is brain cancer.

26. A method of monitoring progression of cancer, comprising
providing a sample containing acellular DNA from a subject suffering from cancer; and
detecting one or more DNA markers in the 12q22-23 region in the sample, wherein LOH of the DNA markers indicates a high probability of a progressing cancer.

27. The method of claim 26, wherein the sample is a serum sample.

28. The method of claim 26, wherein the sample is a plasma sample.

29. The method of claim 26, wherein the DNA markers include D12S1657, D12S393, D12S1706, D12S346, or a combination thereof.

30. The method of claim 26, wherein the DNA markers are associated with the APAF-1 gene.

31. The method of claim 26, wherein the cancer is melanoma.

32. The method of claim 26, wherein the cancer is colon cancer.

33. The method of claim 26, wherein the cancer is breast cancer.

34. The method of claim 26, wherein the cancer is brain cancer.

35. A method of determining the efficacy of a cancer therapy, comprising
providing a sample containing acellular DNA from a subject suffering from cancer and administered with a therapy; and
detecting one or more DNA markers in the 12q22-23 region in the sample, wherein LOH of the markers indicates poor efficacy of the therapy.

36. The method of claim 35, wherein the sample is a serum sample.

37. The method of claim 35, wherein the sample is a plasma sample.

38. The method of claim 35, wherein the DNA markers include D12S1657, D12S393, D12S1706, D12S346, or a combination thereof.

39. The method of claim 35, wherein the DNA markers are associated with the APAF-1 gene.

40. The method of claim 35, wherein the cancer is melanoma.

41. The method of claim 35, wherein the cancer is colon cancer.

42. The method of claim 35, wherein the cancer is breast cancer.

43. The method of claim 35, wherein the cancer is brain cancer.

44. A method of determining the probability of survival, comprising
providing a sample from a subject suffering from a metastatic cancer; and
detecting one or more DNA markers in the 12q22-23 region in the sample, wherein LOH of the markers indicates a low probability of survival.

45. The method of claim 44, wherein the sample is a tumor sample.

46. The method of claim 44, wherein the sample is a serum sample.

47. The method of claim 44, wherein the sample is a plasma sample.

48. The method of claim 44, wherein the DNA markers include D12S1657, D12S393, D12S1706, D12S346, or a combination thereof.

49. The method of claim 44, wherein the DNA markers are associated with the APAF-1 gene.

50. The method of claim 44, wherein the cancer is melanoma.

51. The method of claim 50, wherein the melanoma is a stage III melanoma.

52. The method of claim 51, wherein the melanoma is an RLM melanoma.

53. The method of claim 51, wherein the melanoma is an ITM melanoma.

54. The method of claim 50, wherein the melanoma is a stage IV melanoma.

55. The method of claim 44, wherein the cancer is colon cancer.

56. The method of claim 44, wherein the cancer is breast cancer.

57. The method of claim 44, wherein the cancer is brain cancer.

58. A method of determining the probability of responsiveness to a therapy, comprising
providing a sample from a subject suffering from cancer; and
detecting one or more DNA markers in the 12q22-23 region in the sample, wherein LOH of the markers indicates a low probability of responsiveness to a therapy.

59. The method of claim 58, wherein the sample is a tumor sample.

60. The method of claim 58, wherein the sample is a serum sample.

61. The method of claim 58, wherein the sample is a plasma sample.

62. The method of claim 58, wherein the DNA markers include D12S1657, D12S393, D12S1706, D12S346, or a combination thereof.

63. The method of claim 58, wherein the DNA markers are associated with the APAF-1 gene.

64. The method of claim 58, wherein the cancer is melanoma.

65. The method of claim 64, wherein the cancer is a matastatic melanoma.

66. The method of claim 65, wherein the melanoma is a stage III melanoma.

67. The method of claim 65, wherein the melanoma is a stage IV melanoma.

68. The method of claim 58, wherein the cancer is colon cancer.

69. The method of claim 58, wherein the cancer is breast cancer.

70. The method of claim 58, wherein the cancer is brain cancer.

71. A packaged product, comprising
a container;
one or more agents for detecting one or more DNA markers at the 12q22-23 region in a sample; and
an insert associated with the container and indicating that the sample contains acellular DNA.

72. A packaged product, comprising
a container;
one or more agents for detecting one or more DNA markers at the 12q22-23 region in a sample from a subject suffering from a metastatic cancer; and
an insert associated with the container and indicating that LOH of the markers indicates a low probability of survival.

73. A packaged product, comprising
a container;
one or more agents for detecting one or more DNA markers at the 12q22-23 region in a sample from a subject suffering from cancer; and
an insert associated with the container and indicating that LOH of the markers indicates a low probability of responsiveness to a therapy.


SEQUENCE LISTING (7 sequences)

SEQ ID NO: 1 - DNA, 20 bp, Artificial
  Synthetic oligonucleotide
acatttctcacgatgctacc

SEQ ID NO: 2 - DNA, 18 bp, Artificial
  Synthetic oligonucleotide
caattcatgaagtggcaa

SEQ ID NO: 3 - DNA, 24 bp, Artificial
  Synthetic oligonucleotide
tgctgacaagactgcaaagatctg

SEQ ID NO: 4 - DNA, 20 bp, Artificial
  Synthetic oligonucleotide
gtcgttgttcgagttcggta

SEQ ID NO: 5 - DNA, 20 bp, Artificial
  Synthetic oligonucleotide
gcgtaaaaatacccgcctac

SEQ ID NO: 6 - DNA, 21 bp, Artificial
  Synthetic oligonucleotide
gggtgtgttgttgttgtttga

SEQ ID NO: 7 - DNA, 20 bp, Artificial
  Synthetic oligonucleotide
aaatacccacctaccccaca